(19) United States
(12) Patent Application Publication
(10) Pub. No.: US 2012/0231959 A1
(43) Pub. Date: Sep. 13, 2012

(54) PERSONALIZED MEDICAL MANAGEMENT SYSTEM, NETWORKS, AND METHODS

(75) Inventors: Jeffrey J. ELTON, Concord, MA (US); Jayanthi SRINIVASAN, Pittsburgh, PA (US); Victoria JOSHI, Cambridge, MA (US); Raju KUCHERLAPATI, Weston, MA (US); M. Kathleen Behrens WILSEY, Ross, CA (US)

(73) Assignee: KEW GROUP LLC, Concord, MA (US)

(21) Appl. No.: 13/412,386

(22) Filed: Mar. 5, 2012

Related U.S. Application Data
(60) Provisional application No. 61/449,374, filed on Mar. 4, 2011.

(51) Int. Cl. G06Q 50/22; C40B 60/10; C40B 20/00; C12M 1/40; G06Q 50/24; C12Q 1/68

(52) U.S. Cl. 506/2; 705/2; 705/3; 435/6.12; 435/287.2; 506/38

(57) ABSTRACT

Disclosed herein are systems and methods for the assignment of therapeutic pathways to members of a network of oncology. The systems and methods allow for storage of disparate information in a database and determine a uniform semantics for all of the stored information. In addition, the systems and methods allow for the calculation of treatment pathways based on patient information as well as publicly-available information relating to particular diseases, and for the refinement of those treatment pathways as new information is added. Robot-assisted genomic labs permit automated genetic testing, which is integrated with the system.

### CROSS-REFERENCE TO RELATED APPLICATIONS

The present invention claims priority under 35 U.S.C. §119(e) to U.S. Provisional Application No. 61/449,374 filed on Mar. 4, 2011, entitled “Personalized Medical Management System, Networks, and Methods,” incorporated herein by reference in its entirety.

### FIELD OF THE INVENTION

The invention is generally directed to medicine and healthcare. More specifically, the invention is directed to genomic medicine and personalized medicine.

### BACKGROUND

Genomic and personalized medicine have gained increasing attention since the sequencing of the human genome in the middle of the first decade of the twenty-first century. During this period, improved computer technologies have allowed healthcare providers and researchers to obtain, store, and analyze genomic information. The fledgling field of genomic medicine, namely the use of genomic information to guide medical decision making, has become a key component of personalized medicine.

Personalized medicine is a field of healthcare that deals with the unique genomic, proteomic, and environmental contexts affecting every patient. Genomic tools developed since the determination of the human genome allow for more precise prediction and treatment of disease.

However, personalized medicine will require the development, standardization, and integration of information from several sources into a system that is accessible to healthcare professionals, researchers, and providers. Such information would also include information relating to health risk assessment, family health history, and clinical decision support for complex risk and predictive information. To date, such a system does not exist and this information is spread across multiple databases and systems. Furthermore, these disparate systems do not even share the same semantics and terminology.

Additionally, the present healthcare system suffers from inefficiencies in treatment decisions made by practitioners who lack the breadth of knowledge relating to treatment regimes known in the literature. This lack of breadth leads to potentially wasteful treatments that will have little impact on disease prognosis and could decrease the quality of life for the patient. Conversely, researchers and clinicians may have breadth of knowledge relating to particular treatments, but do not have the depth of knowledge that practitioners have with their patients. This disconnect leads to inefficiencies in the healthcare system that increase costs with little or no benefit to the patient.

Therefore, there remains a need to provide a system that stores the disparate information using uniform semantics so that information is accessible to all members of a network, i.e., clinicians, patients, insurers, practitioners, and researchers. In addition, there remains a need to provide members of a network calculated treatment pathways based on information stored in the system. Such pathways can vastly decrease the cost of treating patients, while increasing the quality of life of the patients and efficiency of treatment. Furthermore, such a system would allow particular patients with certain genetic profiles to be entered into clinical trials, further improving the development of new therapies and improving healthcare provided to patients.

### SUMMARY

Disclosed are systems and methods for the assignment of therapeutic pathways to members of a network of oncology practices. The systems and methods allow for storage of disparate information in a database. The systems disclosed herein further provide a uniform semantics to all of the information. The information is thus accessible to all members of a network, i.e., clinicians, patients, insurers, practitioners, and researchers. In addition, the systems and methods disclosed herein allow for the calculation of treatment pathways based on patient information as well as publicly-available information relating to particular diseases. Such systems and methods, therefore, decrease the cost and inefficiency in the healthcare system.

Disclosed are systems and methods for the assignment of oncology patients to specific clinical episodes of care for purposes of treatment reimbursement. The episodes are defined according to specific criteria of organ system cancer, tumor staging, line of therapy, and other clinical criteria that map to discrete sets of evidence-based treatment pathways (“ETPs”). Bundled payment systems (also known as “case rates” or “episode-based payment”) would make a single payment for all services related to a treatment or condition, possibly spanning multiple providers in multiple settings (e.g., lab, medical oncology, radiation oncology, diagnostic imaging). Clinical episodes define clinical treatment approaches that are pre-authorized for reimbursement by private or public payers subject to demonstrated compliance to the ETPs. Claims data including information from inpatient, carrier, outpatient, home health, SNF, and DME files will be included to determine costs. Reimbursement for capital expenses, education cost and had debt are explicitly excluded. The ETPs used for treatment provide the frame for episodes based upon which a single bundled payment would be established. In addition, the system contains rules for authorization/validation of claims submitted for a patient in an episode. Data from payer is received by the system and processed. The system also allows for payments to be disbursed from the system after a claim is validated.

Aspects of the methods disclosed herein include methods of assigning therapeutic pathways to members of a network of oncology treatment providers. The methods comprise compiling patient data from a network of oncology providers into one or more databases and compiling publicly available information into the one or more databases. In addition, the methods comprise integrating the patient data and publicly available information into a data set having normalized semantics and identifying a pattern from a comparison of the patient data to the publicly available information. The methods also involve calculating a therapeutic pathway based on the pattern, and providing the therapeutic pathway to a user and monitoring the outcomes.

In certain embodiments, the publicly available information is obtained from one or more of clinical trials, university research laboratories, network members, cancer centers, and government research laboratories. In other embodiments, the publicly available information is obtained from one or more of national cancer registries, FDA databases, genomic databases, and databases administered by the National Institutes of Health. In particular embodiments, the publicly-available information is obtained from payers, patient health records, and employers and the publicly-available information comprises information from health record accounts, claim information, self reported information, data related to the clinical information preference, and publicly-available information from other sources. Such publicly-available information, in certain instances, comprises genetic information, phenotypic information, genetic profiles, correlations of genetic profiles to disease phenotypes, disease prognoses for genetic profiles, and therapeutic outcomes determined for available therapies.

Embodiments of this aspect further comprise alerting the network to new information relating to the therapeutic pathway. Other embodiments of this aspect further comprise recalculating the therapeutic pathway for the patient based on the new information. Still additional embodiments of this aspect further comprise tracking compliance of the user to the calculated pathway, calculating the reimbursement of the user based on the compliance with the pathway and/or obtaining a sample from one or more patients and determining one or more genetic profiles of the one or more patients from one or more tissue sites. In certain embodiments, the methods further comprise compiling the one or more genetic profiles into the one or more databases and/or identifying one or more patterns from a comparison of the one or more genetic profiles to the publicly available information. In certain instances, aspects of the methods further comprise a therapeutic outcome of the therapeutic pathway.

In some embodiments, the patient data comprises one or more genetic profiles and the medical histories of the one or more patients. In additional embodiments, the publicly available information comprises clinical research data and/or data obtained from clinical trials.

Additional aspects are disclosed that further comprise updating the one or more databases with the therapeutic outcome associated with the therapeutic pathway, recalculating the therapeutic pathway based on the therapeutic outcome and providing the recalculated pathway to the members, and/or compiling financial data from the user.

In certain embodiments, the financial data is integrated into the data set and in some of such embodiments comprises costs associated with the care of the patient. In such embodiments, the methods further comprise tracking costs associated with the care of the patient. In other embodiments, the calculating of the therapeutic pathway comprises generating an evidence-based treatment protocol. In particular embodiments, the therapeutic pathway guides treatment of one or more patients.

In certain embodiments, the methods further comprise organizing oncology practices into regional networks and/or organizing the regional networks into a national oncology network. In certain other embodiments, identifying a pattern from a comparison of the patient data to the publicly available information comprises recognizing a pattern in the information and associating the pattern with the patient data.

In particular embodiments, the methods further comprise analyzing a DNA sequence for at least one region of DNA from a patient or tissue source obtained from a patient, identifying a variation or set of variations in the DNA sequence of the patient as compared to a reference DNA sequence, and/or further comprising querying the one or more databases to identify evidence establishing a relationship between the variation or set of variations and one or more of a disease, a therapeutic outcome, or a disease prognosis. In more particular embodiments, the methods further comprise generating a hypothesis based on the variation or set of variations and evidence of the relationship, identifying a previously unknown variation or set of variations and compiling these in the one or more databases, producing evidence of a relationship between the unknown variation and one or more of a disease, a therapeutic outcome, or a disease prognosis, and/or providing the evidence to the user. In still more particular embodiments, a plurality of genes are analyzed.

In certain embodiments, the publicly available information comprises the existence of one or more clinical trials testing one or more therapies. Aspects of the methods disclosed herein further comprise identifying one or more clinical trials for which a patient qualifies and/or creating a cohort of patients for inclusion in a clinical trial based on one or more of genetic and phenotypic information stored in the one or more databases. In some aspects of the methods disclosed herein, the one or more databases compile structured and unstructured data. In some embodiments, the databases store digital data that comprise images, sound text, and structured information from electronic medical records.

In certain aspects of the methods disclosed herein, the methods further comprise testing for a mutation in one or more genes in a patient or a tissue source derived from a patient, the mutation having a known effect on one or more treatments, testing for a mutation in one or more genes in a patient, and/or researching the potential effects of the mutation on one or more treatments. In certain embodiments, the mutation has no known effect on a treatment. In still more particular aspects, the methods further comprise compiling patient data from patient health records, payer related data and self reported data.

Disclosed herein are systems for genomic analysis and therapy knowledge management. The systems comprise one or more robot-assisted genomic labs and a database in communication with the one or more robot-assisted genomic labs, the database configured to store patient data obtained from the genomic labs, publicly available information, and patient-centric information. The system further includes a module configured to integrate the patient data and the information into a data set having normalized semantics, a module configured to identify a pattern from a comparison of the patient data and patient-centric information to the publicly available information and a module configured to calculate a therapeutic pathway based on the pattern. As used herein, the term “module” means algorithms, logic, software code (e.g., source code), and other programming used in the performance of particular tasks or functions. The term module is also meant to encompass one or more algorithms or logic used to perform a task. For example, a module can be one or more algorithms performing the tasks disclosed herein. In additional aspects, the system comprises a module configured to display the calculated pathway to a healthcare provider.

Aspects of the system further comprise a module configured to alert the healthcare provider of new information stored in the database relating to the calculated pathway and/or a module configured to track costs associated with care of the patient. In certain embodiments, the system comprises one or more of NoSQL databases, columnar databases, and object databases.

In other aspects, the system further comprises a module configured to display to the healthcare provider information compiled in the database, a module configured to scan the database for the new information, and/or further comprising a module configured to recalculate the calculated pathway based on the new information. Aspects of the system also further comprise a module configured to track compliance of healthcare providers with the calculated pathway and/or a module configured to calculate reimbursements based on healthcare provider compliance with the calculated pathway.

In certain embodiments, the publicly available information is obtained from one or more of clinical trials, university research laboratories, network members, cancer centers, and government research laboratories. In particular embodiments, the publicly available information is obtained from one or more of national cancer registries, FDA databases, genomic databases, and databases administered by the National Institutes of Health. In still other embodiments, the publicly available information comprises genetic information, phenotypic information, genetic profiles associated with one or more diseases, correlations of genetic profiles to phenotypes, disease prognoses, and therapeutic outcomes determined for available therapies. In further embodiments, the patient-centric information comprises health reimbursement accounts, electronic medical records, patient health records, a personal medical history, and family history of the patient. In still other embodiments, the publicly available information comprises genetic and phenotypic information, genetic profiles associated with one or more diseases, correlations of the genetic profiles to prognoses, correlation of genetic profiles to therapeutic outcomes, drug label warnings, and clinical research data.

Aspects of the system further comprise a module configured to devalue information that is determined to be of lower relevance to the genetic profile or the calculated pathway than other information in the database, a module configured to analyze DNA sequence information generated by the robot-assisted genomic labs, and/or a module configured to generate patient data based on the DNA sequence information. In certain embodiments, the a module configured to calculate the therapeutic pathway generates an evidence-based treatment protocol. In particular embodiments, the patient data comprises a genetic profile.

Aspects of the system further comprise compiling the genetic profile into the database and/or compiling financial data from the healthcare provider. In certain embodiments, the financial data is integrated into the data set. In some embodiments, the financial data comprises costs associated with the care of the patient.

Aspects of the system further comprise a module to compare the patient data and the patient-centric information to the publicly available information stored in the database, a module configured to recognize a pattern in the publicly available information and associate the pattern with the genetic profile of the patient, and/or a module configured to identify a variation or set of variations in a DNA sequence of a patient or a tissue obtained from the patient as compared to a reference DNA sequence. Other aspects of the system further comprise a module configured to query the database to identify evidence establishing a relationship between the variation or set of variations and one or more of a disease, a therapeutic outcome, or a disease prognosis, a module configured to generate a hypothesis based on the variation and evidence of the relationship, and/or a module configured to identify a previously unknown variation or set of variations and to compile the variation in the database. Still other aspects of the system further comprise a module configured to produce evidence of a relationship between the unknown variation and one or more of a disease, a therapeutic outcome, or a disease prognosis. In certain embodiments, the publicly available information comprises an existence of one or more clinical trials testing one or more therapies.

Aspects of the system further comprise a module configured to identify one or more clinical trials for which a patient qualifies and/or a module configured to create a cohort of patients for inclusion in a clinical trial based on one or more of genetic and phenotypic information stored in the one or more databases.

Aspects of the methods disclosed herein are directed to methods of calculating a therapeutic pathway for a patient suffering from a disease. The methods comprise generating a genetic profile of the patient or a tissue source obtained from a patient and compiling the genetic profile and a medical history of the patient into a database. The methods also comprise compiling publicly available information into the database and comparing the genetic profile and the medical history of the patient to the information compiled in the database. The methods further comprise identifying a pattern in the publicly available information and associating the pattern with the genetic profile of the patient and calculating the therapeutic pathway based on the pattern identified from the comparison. The methods also comprise providing the pathway to a user, the therapeutic pathway guiding treatment of the disease.

In certain embodiments, the therapeutic pathway comprises one or more suggested actions predicted to be more likely to yield a positive and cost-effective outcome for the patient. In some embodiments, the publicly available information is obtained from one or more of national cancer registries, FDA databases, genomic databases, and databases administered by the National Institutes of Health. In other embodiments, the publicly-available information comprises genetic and phenotypic information, genetic profiles associated with one or more diseases, correlations of the genetic profiles to prognoses, correlation of genetic profiles to therapeutic outcomes, drug label warnings, and clinical research data.

Certain aspects of the methods further comprise monitoring the therapeutic outcome of the therapeutic pathway, compiling new information relating to the therapeutic pathway into the database, and/or recalculating the therapeutic pathway based on the new information. Additional aspects of the methods further comprise alerting the user to the recalculated therapeutic pathway and/or monitoring compliance with the therapeutic pathway. Some aspects of the methods further comprise determining whether a genetic profile contains a mutation that is associated with a pathological condition or is benign.

In certain embodiments, the medical history of the patient comprises the family medical history and the treatment history of the patient. In particular embodiments, the user is a healthcare provider. In still other embodiments, the therapeutic pathway comprises an evidence-based treatment protocol. In further embodiments, the disease is a cancer.

### DESCRIPTION OF THE FIGURES

The following figures are presented for the purpose of illustration only, and are not intended to be limiting:

FIG. 1 shows an exemplary genomic analysis and therapy knowledge management system.

FIG. 2 is a diagrammatic representation showing therapeutic pathway options provided to a patient diagnosed with non-small cell lung carcinoma

FIG. 3 is a diagrammatic representation of a robot-assisted genomic lab for genomic processing (i.e., the isolation of nucleic acids from a sample and the sequencing of the nucleic acids) of biological samples obtained from patients.

FIG. 4 is a representation of an exemplary method of calculating a therapeutic pathway for a patient being treated by a practitioner at one of the members in the network.

FIG. 5 shows a method of identifying a therapeutic pathway for a patient being treated by a practitioner at one of the members of the network. In this method, the system obtains a biological sample and processes the sample.

FIG. 6 shows a methodology by which financial information is stored in the system disclosed herein and utilized by member healthcare providers to determine reimbursement and compliance with evidence-based treatment protocols.

FIG. 7 shows the process by which information relating to treatment errors for various cancers is compiled into the one or more databases of the system. This information is also used to aggregate and compute financial data (e.g., transaction and drug data).

FIG. 8 is a screen capture showing the evidence-based treatment protocol for a patient having non-small cell lung carcinoma (“NSCLC”). The protocol shows the best protocol for treatment of the disease based on Genomic Test Results.

FIG. 9 shows the information stored in the system. The information relates to particular patients (“Patient Name”) and the pathways provided for each patient (“Requested Regimen”). Also provided in the database is the particular practice treating each patient.

FIG. 10 shows an example of the information that is provided to the system from an electronic health record.

FIG. 11 is a graphical representation showing the organization of a network system. The healthcare practice member and system with database are linked to payers that reimburse the healthcare provider for services.

FIG. 12 is a representation of a worksheet showing information obtained by a healthcare provider and provided to the system for storage in one or more databases.

FIG. 13 is a screen capture showing the information stored in the database and provided when accessed by a user and/or network member. The screen capture includes information relating to a particular cancer center (“Practice”), cancer locations/types (“Dx Group”), stages (“Stage”), and treatment regimens (“Regimens”).

FIG. 14 is a graphical representation showing the information provided by a healthcare provider and its connection to the knowledge management system.

FIG. 15 is a screen capture of a portal providing a healthcare provider with information relating to particular clinical trials. The screen capture shows that information for clinical trials to which the patient could be eligible.

### DETAILED DESCRIPTION

All publications, patent applications, patents, and other references mentioned herein are incorporated by reference in their entirety. In case of conflict, the present specification, including definitions, will control. In addition, the materials, methods, and examples are illustrative only and not intended to be limiting.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### 1. DEFINITIONS

For convenience, certain terms employed in the specification, examples and claims are collected here. Unless defined otherwise, all technical and scientific terms used in this disclosure have the same meanings as commonly understood by one of ordinary skill in the art to which this disclosure belongs. The initial definition provided for a group or term provided in this disclosure applies to that group or term throughout the present disclosure individually or as part of another group, unless otherwise indicated.

The articles “a” and “an” are used in this disclosure to refer to one or more than one (i.e., to at least one) of the grammatical object of the article. By way of example, “an element” means one element or more than one element.

A “genetic profile” is information relating to the nucleic acid sequence (DNA or RNA) or epigenetic changes to this DNA or RNA of one or more biomarkers in a patient's genome. The biomarkers are typically known to be correlated with or hypothesized to be correlated with a certain disease state, prognosis, or treatment regime. In addition, the genome can be a germline, a tumor genome, a genome of a metastatic cell, a genome of a microbe infecting the individual, or a genome of a cell containing a mutation.

“Evidence-based treatment protocol” is a set of therapeutic procedures that is determined from published and non-published information. “Evidence-based treatment protocols” are established as authoritative either by their statistical validity, experts in the field making such evaluations and determinations, the quality of the scientific and clinical journals publishing the results, and/or the number of independent determinations of said outcome. The information is obtained from sources such as publicly-available sources (e.g., clinical research, research literature, and databases), diagnostic procedures performed on a patient, and the family history of a patient.

As used herein, a “therapeutic pathway” is a clinical treatment plan that includes diagnosis of disease, molecular characterization of the disease, and/or therapeutic regimens including drug selection, dosing, and/or schedules.

### Genomic Analysis and Therapy Knowledge Management Systems

The disclosure provides, in part, genomic analysis and therapy knowledge management systems for assigning therapeutic pathways to patients in members of a network. The network can comprise healthcare providers that treat a particular disease or diseases. For instance, the network can comprise oncology treatment providers.

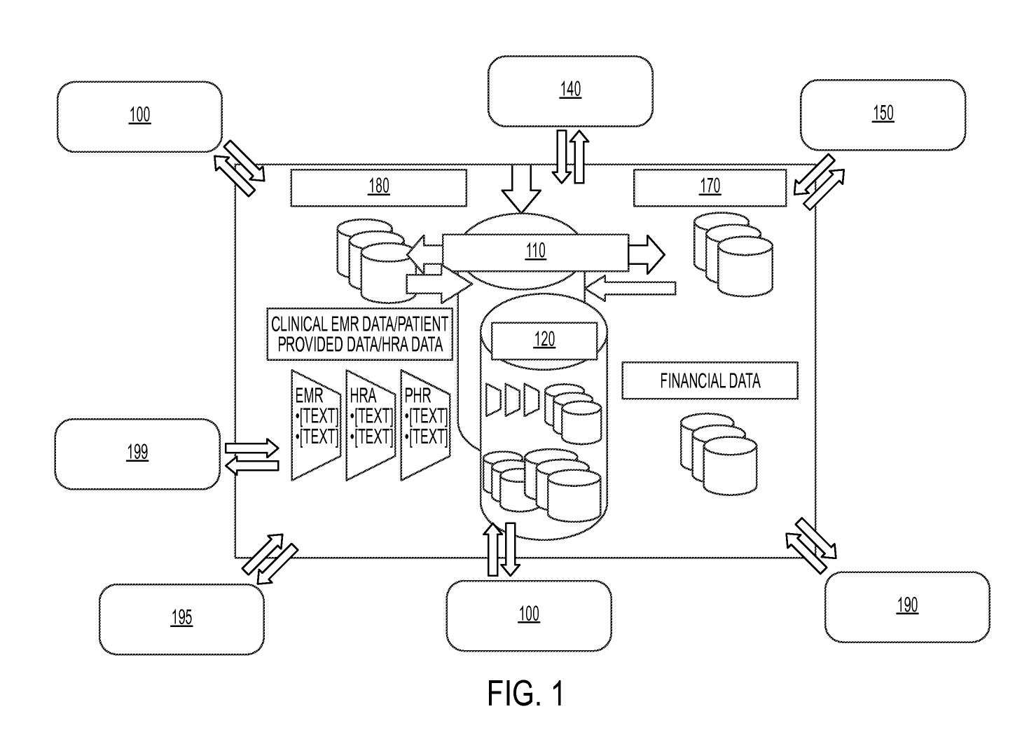

An exemplary genomic analysis and therapy knowledge management system is shown in FIG. 1. Patient data 100 is received from practitioners (i.e., physicians and other healthcare providers). One type of patient data is “patient-centric information,” which includes health reimbursement accounts, electronic medical records, patient health records, personal medical history, and family history of the patient. The arrows represent links between the practitioners and the system, the links allowing communication between the system and the practitioners. It is understood that by “links” is meant any physical connection or non-physical connection that allows transmission of information. Physical connections include optical fiber, coaxial cable, twisted pair or otherwise, and non-physical connections include “wireless” connections, such as cellular, microwave, IR, laser or any other connection that does not require a wire. Members of the network can access patient data and information stored in the database through the links.

In addition to patient data being received by the system, the system receives and stores publicly-available information from regulatory agencies 120 such as the FDA and NIH. Other publicly-available information is received from researchers 130, pharmaceutical companies 140, and clinical researchers 150. In many instances, publicly available information is obtained from clinical trials, university research laboratories, network members, cancer centers, national cancer registries, FDA databases, genomic databases, databases administered by the National Institutes of Health and government research laboratories. “Publicly-available information” includes information that is relevant to the prognosis of a disease, the onset of a disease, and the response a disease will have to treatment. Such information also includes genetic and phenotypic information, genetic profiles associated with one or more diseases, correlations of the genetic profiles to prognoses, correlation of genetic profiles to therapeutic outcomes, drug label warnings, and clinical research data. In each case, the information can be obtained directly from the sources over the internet or by having the information input into the system from a personal computer or other machine linked to the system. The information can also be obtained from clinical trials organized by members of the network. Other information also includes electronic medical records, personal health accounts, and health reimbursement accounts.

Once the information is received by the system, it is stored in one or more databases that compile patient data. Exemplary database technologies include Parracel, columnar databases, Oracle Terradata, and NoSQL databases. In FIG. 1, the database is separated into several components. The system comprises an operational data store 110. The operational data store integrates the information using module configured to integrate the patient data and the information into a data set having normalized semantics. The system also allows clinicians, researchers, and other members of the network to access the integrated information. In this embodiment, the patient data 100 and publicly-available information is integrated into a data set having normalized semantics. The system normalizes the semantics of the disparate information by establishing semantic alignment between underlying data structures from the disparate information sources. Such algorithms useful for semantic alignment are known in the art and include Orion, Amalga and Dbmotion address facets of semantic integration. In addition, Intelligent Medical Objects (IMO) and Health language and Mmodal are also useful in semantic interoperability. In addition, the operational data store 110 handles queries on small amounts of data. The operational data store 110 also acts to store information for short periods of time prior to storing the information in the data warehouse 120.

Furthermore, the system handles several types of semantic integration for a common set of terms for test, diagnosis treatment. The system allows for data to be interpreted and aligned no matter its source. In certain embodiments, this is accomplished by providing normal ranges for labs for each lab data so the information can be compared. For example, the system can identify results in the database in ICD-9-CM and SNOMED-CT—both of which are different clinical code terminologies—relating to different parameters such as “body structure” and “clinical finding.” In an exemplary embodiment, the clinician queries the system for results relating to leiomyoma. The system searches for data stored in ICD-9-CM and SNOMED-CT. The system provides the information to the clinician from the database. In other embodiments, all data is converted to a common format and then displayed to show a normalized set of data that is comparable to each other.

In some embodiments, the system further comprises an enterprise data warehouse 120. The data from the operational data store 110 is stored and cataloged in the enterprise data warehouse 120. The data warehouse 120 also has the capability to integrate the information into a data set having normalized semantics and allows access to the data so that it performs all of the functions of the operational data store 110. The data warehouse 120 can include a single computer or many computers (e.g., servers), and associated storage. In some embodiments, the data warehouse 120 has module allowing it to connect to the operational data store 110 and to other computers connecting to the system, including computers located at members of the network. Alternatively, the data warehouse 120 can connect to a server layer providing connectivity to network members.

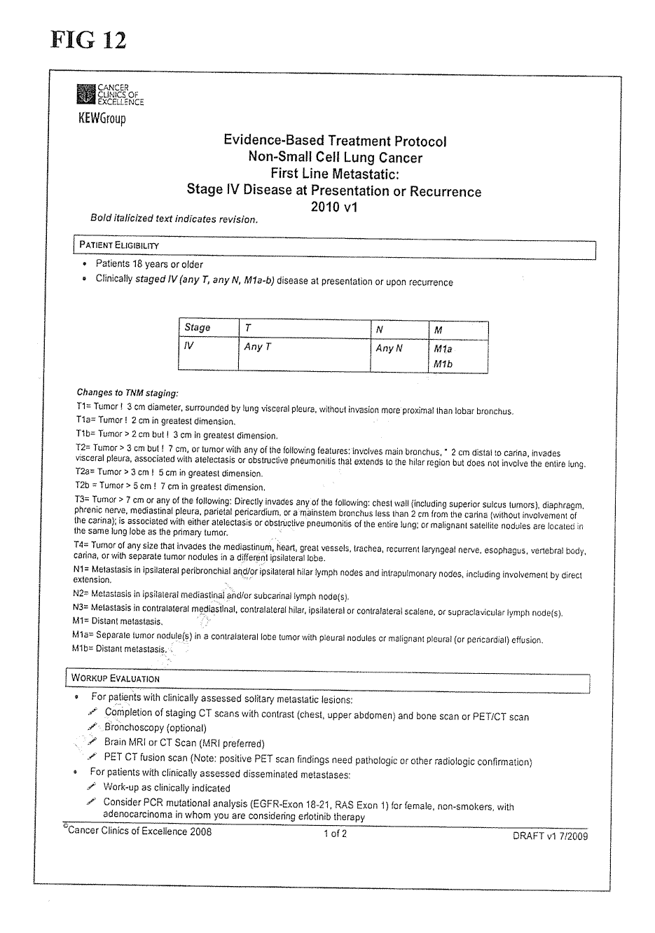

Furthermore, the system stores proven evidence-based treatment protocols 170. FIG. 12 shows an example of the information obtained to develop an evidence-based treatment protocol. The information shown relates to a patient having non-small cell lung carcinoma. The information is collected by a healthcare provider and is utilized to determine the proper protocol for a particular patient based on the information obtained during a patient checkup. In FIG. 12, the evidence-based treatment protocol relates to non-small cell lung carcinoma in which a patient has been diagnosed with Stage IV cancer.

Evidence-based treatment protocols can be either “proven” or “hypothetical.” By “proven” is meant that the evidence-based treatment protocol has information supporting the protocol. The system also stores hypothetical evidence-based treatment protocols 180. By “hypothetical” is meant that the evidence-based treatment protocol does not yet have information, or has limited information, supporting the hypothesis generated by the system. There are different criteria by which a protocol can be labeled “proven.” In certain embodiments, a protocol is recommended by the drug label. In some instances, a professional organization has developed a policy regarding treatment based on evidence accumulated by the organization. In particular embodiments, an assessment of published data from clinical trials is performed and the evidence is utilized to move a pathway from “hypothetical” to “proven.” In particular embodiments, evidence-based treatment protocols are based on the work of teams of experts evaluating the results of published and unpublished clinical studies based on “strong-form” statistical validity or the work of multiple centers with independent, comparable results. A proven protocol is one which has been ratified by internal clinical experts based on evidence and cumulative information derived from literature, clinical research, national society guidelines, and researchers as well as other leaders in the clinical field. “Hypothetical” treatment protocols are generated by individuals, and are often extensions of proven protocols. For example, a treatment is known to be effective in breast tumors with a certain genetic composition, and a hypothetical treatment could be that the same treatment is effective in lung tumors with the same genetic composition. As such, “hypothetical” treatment protocols can be utilized when there are no treatments available for a particular disease or subset of disease, but there are treatments available for diseases having similar or related genetic compositions (as in cases were components of similar genetic pathways are affected) or phenotypic characteristics. A hypothetical protocol is based on observance of a pattern of recurring correlations. Hypothetical treatment protocols are subject to validation based on increasing levels of clinical evidence coming from published studies and cumulative treatment results observed and stored in the system. A hypothesis is tested to prove the correlation and adequately prove the association between related observances. The hypothesis is refined through testing to create a clinical trial protocol. This clinical protocol can be validated through the traditional clinical trial process or through retrospective analysis of data in the system depending on the type of hypothesis being considered.

In certain embodiments, the process of moving a treatment protocol from “hypothetical” to “proven” involves multiple steps. For instance, there could be evidence based on a FDA approved drug, diagnostic methodology, or treatment, e.g., KRAS for colon cancer, BCR-ABL for CML, and Her2 amplification for breast cancer. The treatment is eventually supported by evidence such as practices in major medical centers and approval of a treatment or diagnostic in the European Union. Additional evidence can be accumulated into the system such as data from late stage clinical trials.

As shown in FIG. 1, the system also stores other financial data and other information, such as payer data 190, care coordinator information (e.g., account numbers) 195, and information relating to physicians 199. All such information can be stored in separate servers from the data warehouse 120 or stored directly into the data warehouse 120.

The system depicted in FIG. 1 also comprises module configured to identify a pattern from a comparison of the patient data and patient-centric information to the other information (e.g., publicly available information) stored in the operational data store 110 and/or the data warehouse 120. In certain embodiments, the patterns are identified by a comparison of the patient data 100 to the information obtained from researchers 130, pharmaceutical companies 140, and/or clinical researchers 150. Algorithms for identifying patterns in structured and unstructured data are known in the art. Exemplary algorithms useful for pattern recognition include probabilistic context free grammars, bootstrap aggregating, boosting, and ensemble averaging. Additional algorithms include algorithms designed based on Neural networks, pattern recognition, geometrical analysis of data, and dynamic maintenance of semantic ontologies. Also, a range of AI techniques (learning, ontologies, inferencing) can organize and derive useful information from the huge base of medical information in science and business publications, professional journals, clinical test reports. Such algorithms include Autonomy, Anvita, attensity, atigeo, mmodal, medstor, and Aysadi.

The system comprises modules that allow for comprehensive real-time analytics involves collecting and using clinical, genetic financial data to enhance patient care, cost, safety and efficiency, and data are examined on a variety of levels including the ability to interpret clinical data at the point of decision making. The modules are configured to evaluate a specific patient or member, evaluate a population, evaluate a specific provider or provider network, evaluate prevalence or treatment of a specific condition, evaluate an episode of care with respect to Cost quality and efficacy, evaluate clinical trials, and extract hypotheses to lead to the formation of clinical trials. However, other algorithms known to those of ordinary skill in the art are within the scope of the disclosed systems.

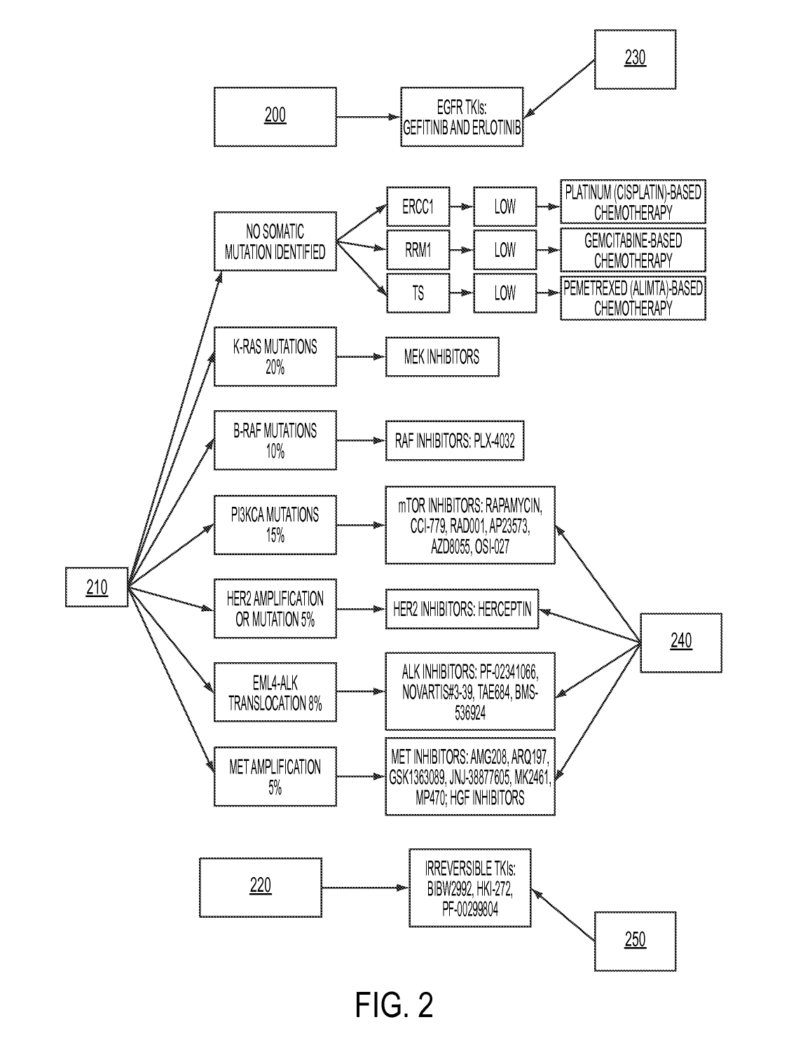

The system utilizes the patterns recognized in the data stored in the operational data store 110 and/or data warehouse 120 to calculate a therapeutic pathway. The system comprises module configured to calculate a therapeutic pathway based on the pattern. Therapeutic pathways are generated based on patient data 100 and the other available information stored in the operational data store 110 and/or data warehouse 120. In certain embodiments, the therapeutic pathway is a decision tree that takes into account both the genotype and phenotype of the patient, as well as data in the database associated with the particular disease of the patient. As shown in FIG. 2, a patient diagnosed with non-small cell lung carcinoma presents with an EGFR sensitizing mutation, identified by genetic analysis of the cancer cells by the diagnostic laboratory. The system contains information relating to non-small cell lung carcinoma involving the effect on therapy of three outcomes of EGFR diagnostic analysis: an EGFR sensitizing mutation 200, absence of EGFR mutation 210, and an EGFR resistance mutation 220. Based on the information in the database for EGFR sensitizing mutations 200, the system calculates that the best treatment for this type of mutation is one of the EGFR TKI, gefitinib or erlotinib, and provides this information to the practitioner. In other words, this information allows the system to generate a range of potential treatment options. In each case, one or more pathways 230, 240, and 250 for potential treatment may be suggested depending on the information. Note that the treatment options in this embodiment involve pharmaceutical courses of treatment. The system can also suggest changes in lifestyle such as changes to exercise habits, cessation of smoking, and dietary changes to improve the quality of treatment.

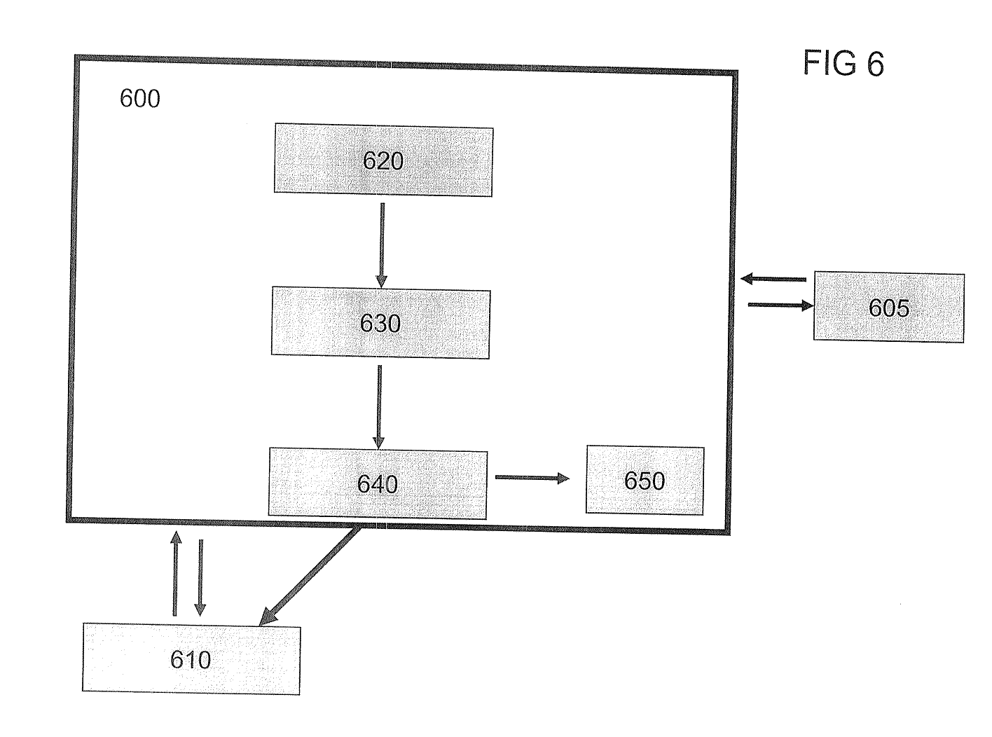

As described above, the system can store financial information from healthcare providers, insurers, and other members of the network. The financial information includes cost of treatment, copayment information, reimbursements, and other financial information relevant to the care of a patient. In these embodiments, the systems further comprise module for tracking the costs associated with the care of a patient. FIG. 6 shows that financial data from various providers is stored in the system 600. The system comprises data connections (e.g., data pipes) 605 to other databases and medical systems to obtain medical information for systems such as Varian Medical Systems (Palo Alto, Calif.) and Impact Medical Solutions. The dataset is normalized and provided to network members. Once the dataset has been normalized, the system develops evidence-based treatment pathways, which are provided to healthcare providers 610. The system monitors compliance with the pathway 620 and determines the costs associated with the pathway 630. The cost information is used, along with success of treatment, by the system to determine whether the pathway is the most cost-effective and treatment effective pathway 640. This information is used to monitor the healthcare provider compliance 650 and is provided to the healthcare providers 610.

The system uses Pay-for-Performance (P4P) program management, physician compliance/efficiency and patient profiling, population health management and outreach, drug substitution, and episode cost/payment to during the analysis of the pathway to use and the proper reimbursement. The system also uses companies such as Treosolutions, Medeanalytics and Medventive to provide alignment of payment to reduce variation, including applying risk-adjusted tools, Linking payment and quality, and patient-centered episode and bundled payments.

In addition, when exceptions to a therapeutic pathway are identified, alerts are sent to individual clinicians, physician practices, designated agents within healthcare practices with responsibility for monitoring compliance and remedying exceptions, and to the team leader responsible for that pathway. In certain embodiments, all requests for payment (claim) by the healthcare provider are sent through the one or more databases for verification of consistency with evidence-based therapeutic pathways. Upon positive verification, the submitted claim is transmitted to the appropriate payer (private insurance company, authorized third party administrator, government entity, or party handling claims processing on behalf of the government entity). No further authorization of payment is required for position confirmation of compliance to the pathways implicated for individual patients within the episode. Incentives for clinicians is based on (a) removing waste/costs, e.g., drug substitution for more efficacious and lower cost and (b) enhanced quality criteria and goals, e.g., compliance threshold. Such module allows for the healthcare provider or insurer to determine the costs associated with a particular treatment and potentially more cost-efficient treatments for a particular disease. The system can integrate the financial information (i.e., financial data) into the data set.

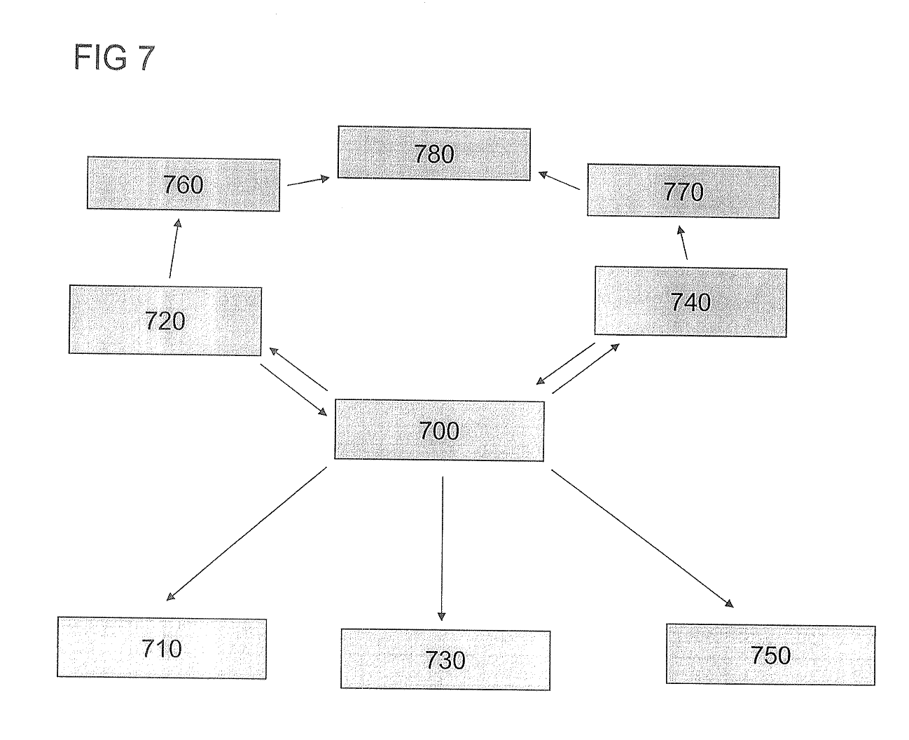

In a specific example of the algorithm used in certain embodiments of the disclosed systems, the system compiles information relating to errors in failed colon, lung, and breast cancer treatment plans 700 (FIG. 7). The system compiles all failed treatments utilizing the evidence-based treatment plans 710, 730, and 750. As an initial step, the system processes treatment rules based on the therapeutics pathways 720 and 740. This information is utilized to compute financial transaction data relating to the costs of treatment 760 and 770 The information is provided to an archive in the one or more databases of the system 780.

The system can also provide alerts or updates to members of the network of new information that is stored in the database relating to therapeutic pathways provided to the practitioners at the member healthcare providers. The system comprises a module configured to scan the database for the new information. For example, if it is proven that patients B-RAF mutation will benefit from PLX4032 then the cohort of patients who can be treated and their physicians are alerted simultaneously. In the example, a clinical trial to use a cocktail of drugs to treat a condition is designed for patients with a specific mutation and this information is in the system. The system identifies the information sends an alert to the patient to contact her physician regarding the clinical trial. Algorithms used for this utilize fuzzy logic, spectral algorithms, and/or other clustering techniques to determine the cohort of patients or clinicians who are impacted by the new information. Upon or after receipt of the new information, the system recalculates the therapeutic pathway and provides the new pathway to the practitioner at the member healthcare provider.

For example, if a genetic variation that makes the drug stay in the body longer than usual, which can cause serious side effects, then discovery of that information, will cause the physician to readjust the dosage of the medication of the patient who has that genetic variation according to a new evidence-based protocol. The recalculated pathway can be used to revise treatment or to refine treatment. For instance, new information relating to the dosage of a drug when prescribed in combination with another drug can lead to a reduction or increase in the dosage of the drug. The system utilizes a rules-based engine that assesses and identifies the most appropriate evidence-based treatment pathway for each individual patient based on data extracted from the electronic medical records and available from the diagnostic image repository or laboratory information system of a centralized molecular diagnostics lab. Based on the most appropriate pathway selected actual clinical activities performance and planned are evaluated for consistency with the criteria established in the evidence-based treatment pathway. In addition, each decision and action taken in a patient's care for compliance with evidence-based treatment pathways is monitored by the rules-based engine.

In certain instances, a therapeutic pathway based on evidence does not exist. In these cases, the system utilizes available information to create new hypothesis or validate an existing hypothesis. A hypothesis is first generated by one or more clinicians or researchers in a network. The hypothesis is provided to the system and stored in the one or more databases. The hypothesis is generated by comparing a patient profile to evidence-based treatment guidelines. If a match is found within the guidelines, then the evidence-based treatment pathway is selected from the guidelines. The patient profile is further checked against a hypothesis set that is generated by the system. If no match is found in the hypothesis set, the patient is treated based on the previously identified pathway. If a match is found, the patient is determined to be potentially relevant to a clinical trial and is identified. The hypothesis is associated with clinical trials and a determination is made as to whether the patient matches the criteria to be enrolled in the identified clinical trial.

As described herein, the system can act as a data repository. Information relating to clinical trials is stored in the one or more databases of the system. The system also monitors the progress of the clinical trials, provides safety review and data review, and allows for the access to information in the one or more databases. Patient data can be collected by the system via phone, device, or the Web.

In the embodiments of the system disclosed herein, algorithms and services from hosted or licensed software are utilized clinical trial monitoring and data capture. Examples of such algorithms include EDC companies such as Medidata, Datatrak, BioClinica (OmniComm, Nextrials, and KIKA. In addition, EPRO solutions include Quintiles' ePRO partner Invivodata with PHT and CRF Health, Merge Healthcare, and Exco InTouch. The system also provides CTMS solutions such as Bioclinica (i.e., TranSenda, Phoenix Data Systems), DZS Software Solutions (i.e., ClinPlus), eResearch Technology, IBM Cognos Clinical Trial Management, Lifetree Research, Medidata Solutions Worldwide, Mednet Healthcare Technologies, Merge Healthcare, OmniComm, Perceptive Informatics (TrialWorks), SAS Institute, and Study Manager.

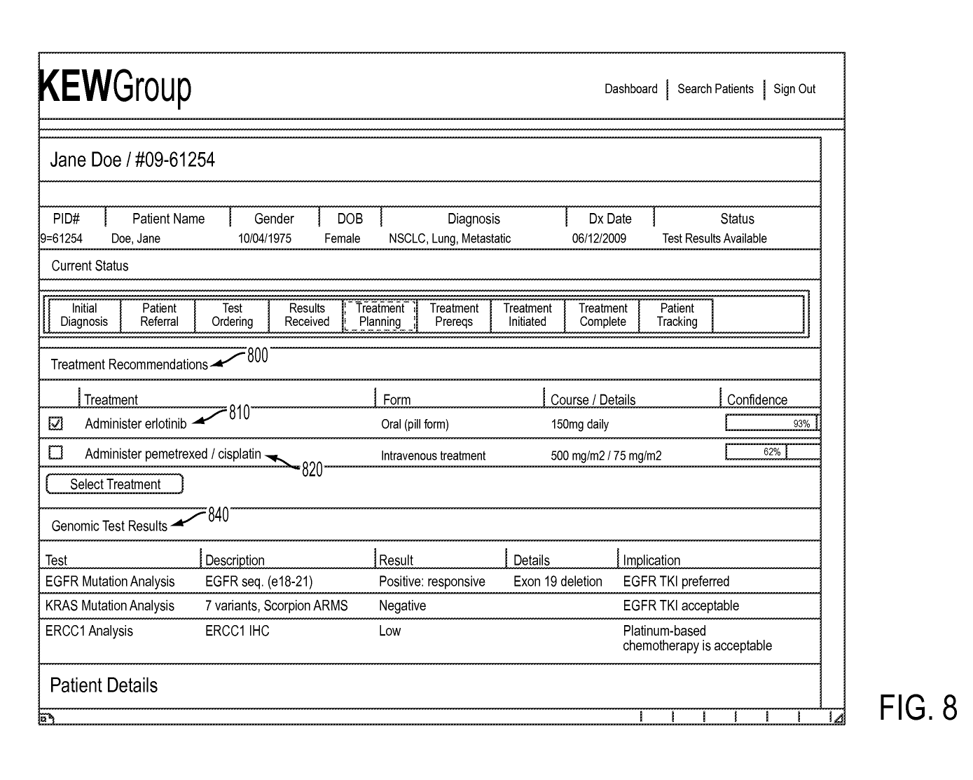

FIG. 8 shows a screen capture of the information presented on the user interface. Patient Jane Doe and all of her vital information are shown. The “Treatment Recommendations” section 800 presents two different pathways for treatment: one utilizing oral erlotinib 810 and the other utilizing IV administered pemetrexed/cisplatin 820. The recommended course of action is erlotinib 830. Each recommendation includes drug form and dosage. Furthermore, the Genomic Test Results section 840 provides the information compiled from testing of the patients genomic samples. The genomic results also indicate the “preferred” and “acceptable” therapies.

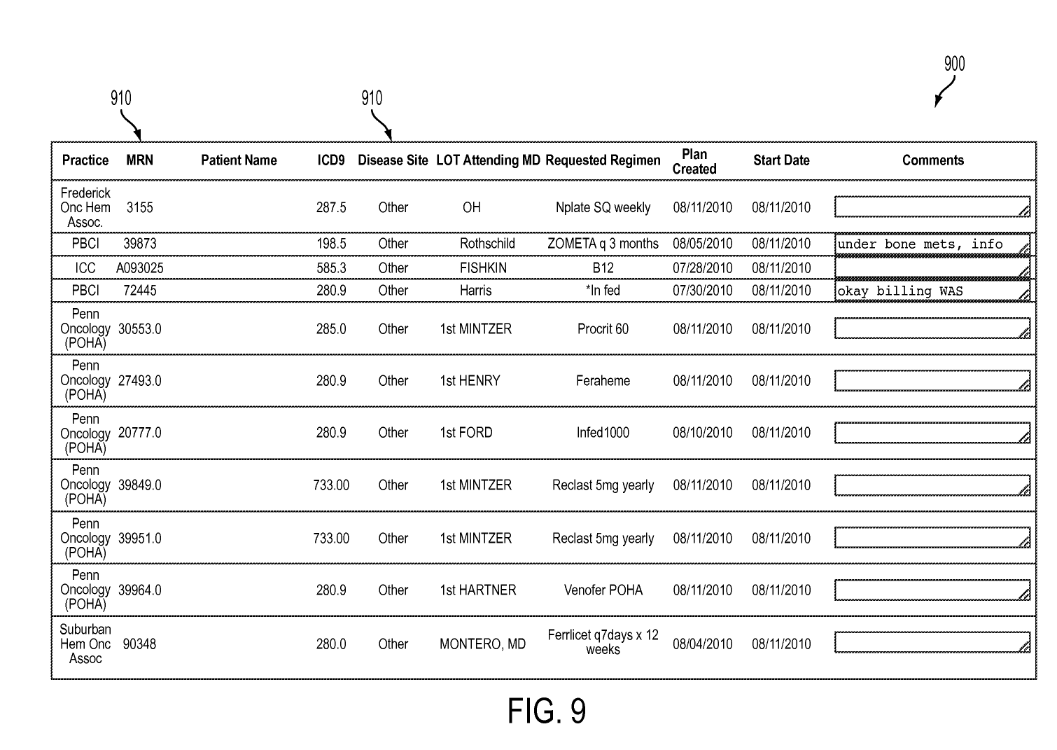

FIG. 9 shows the selected pathways and data associated with particular patients and physicians. The screenshot 900 shown in FIG. 9 contains a toolbar 910. The toolbar 910 contains information relating to the patient, clinical practice of treatment or diagnosis, the disease site, and the treatment plan. The system also allows physicians to enter comments in the system.

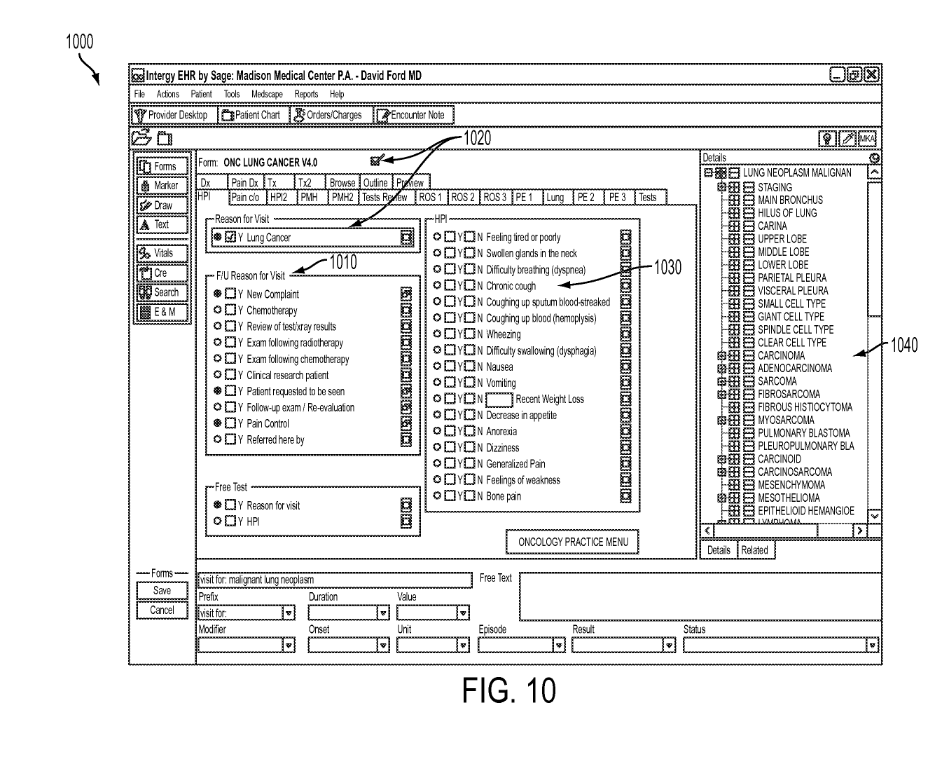

FIG. 10 is an example of the information that is provided to the system from an electronic health record. The electronic health record 1000 captures the information generated during a patient visit to a healthcare provider. The information is entered into the electronic health record 1000 and the electronic health record is subsequently stored in the one or more databases of the system. The system provides detailed information from the EMR, including the reason for the visit 1010, the health state of the patient (in this case, the patient has lung cancer) 1020, the patient complaint 1030, and the type/location of the cancer 1040.

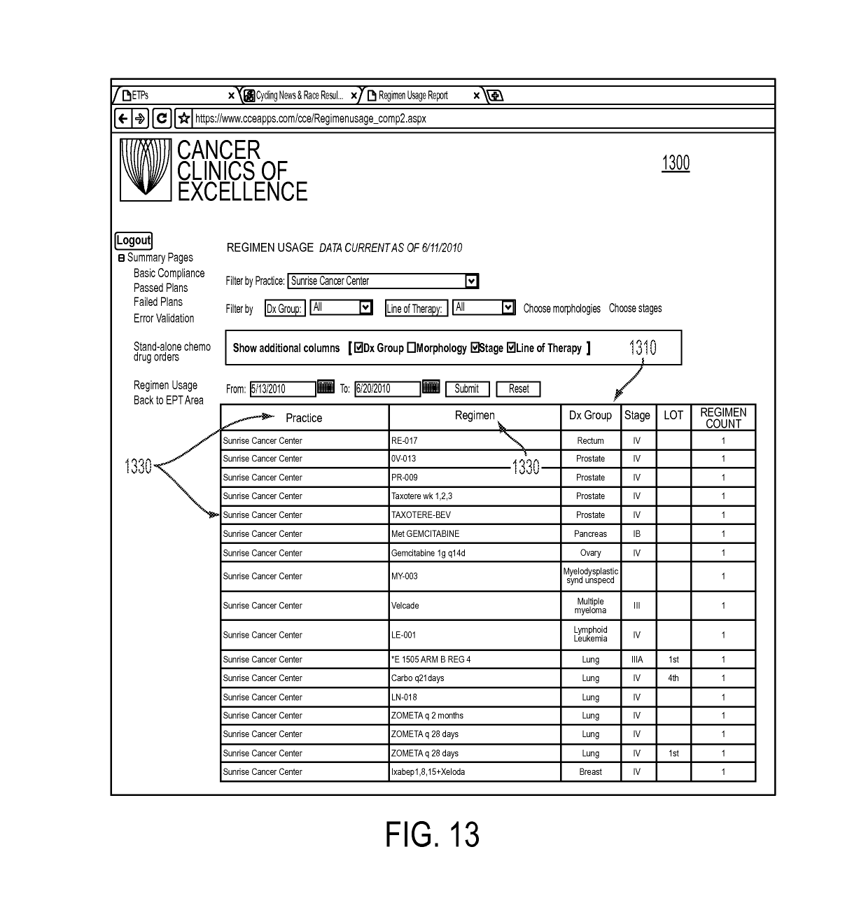

FIG. 13 shows a screenshot 1300 of the type of information stored and tracked by the system. In the screen capture, information relating to particular cancers (“Dx Group”) 1310 is stored from a network member (“Sunrise Cancer Center”) 1320. For each cancer, a stage of the cancer is stored as well as the selected treatment protocol (“Regimen”) 1330.

In a particular example of hypothesis generation, substantial data is generated from clinical trials showing small molecule EGFR TKIs are effective in lung tumors with missense activating mutations in EGFR. Hypotheses could include: (1) that small molecule EGFR TKIs are effective in breast, colon, pancreatic, etc. tumors with missense activating mutations in EGFR and (2) that small molecule EGFR TKIs are effective in lung or other tumors with other mutation types as long as they are predicted to lead to activation the EGFR pathway such as gene amplification of EGFR or activation of downstream components of the EGFR pathway.

In some embodiments, a hypothesis is generated by oncology and genetics experts based on the information stored in the system. For example, some therapeutics target downstream components of fairly linear signal transduction pathways. A hypothesis is generated that if any upstream component of such a pathway were activated in the tumor, and this activation was driving tumorigenesis, inhibition of a downstream effecter would prevent tumor growth. If this hypothesis is proven with strong clinical data for some components of the pathway, this results in a strong association of a genetic variation with a therapeutic pathway. This association is stored in the database and new therapeutic pathways are generated.

There are also embodiments in which the system comprises a module for entering a patient into clinical trials where no therapeutic pathway exists for a particular patient genetic profile. The system has information relating to clinical trials stored in the enterprise data warehouse 120 of FIG. 1. Based on the known requirements to enter the clinical trial, the system will enter the patient into the trial if the patient qualifies. Inclusion and exclusion criteria are defined for each clinical trial. The system stores such criteria directly from enrollment sites that are part of the network and/or from clinicaltrials.gov (registration on this website is currently required for all clinical trials in the US) or other sources known to those of skill in the art. The inclusion and exclusion criteria for each trial will be aligned with fields captured in the electronic health records, such that if all fields have favorable values, the clinical trial will be presented to the provider.

In further embodiments, certain members of the networks are pharmaceutical companies seeking to start and manage clinical trials to test the efficacy and safety of new chemical entities. In these embodiments, the system contains a module to identify potential patients for clinical trials and to create a cohort of patients for inclusion in a clinical trial based on one or more of genetic and phenotypic information stored in the one or more databases. The system looks for criteria whereby the patient may have higher therapeutic benefit for being considered for a clinical trial versus the current best evidence-based treatment approach. Higher therapeutic benefit can be calculated on the basis of current general patient response to the best evidence-based treatment versus the presence of a highly specific genetic mutation in the patient's cancer, or cancers, for which there may be an available clinical trial therapy. The members interested in running clinical trials will access the database and obtain the cohorts of patients. The system also comprises a module to contact the patient's healthcare provider of the patient's qualification for entry into clinical trials if the benefit of entering a clinical trial is determined to outweigh the benefit of the best evidence-based treatment approach. In further embodiments, the system acts as a contract research organization, organizing the patient cohorts, managing the clinical trials, and providing the data to the interested members of the network. In one embodiment, physicians work through a centralized Industrial Review Board (“IRB”) for prospective clinical trial participation.

Upon IRB approval, clinical trials accessible to the practice network are entered into centralized clinical trial database defining patient inclusion and exclusion criteria for involvement. A clinical research rules engine comparable to that used for assessing patient eligibility for evidence-based treatment pathway adapts the trial structure such that individual patients can be automatically assessed for eligibility based on the inclusion and exclusion criteria. New patients entering the database are automatically assessed for their fit against the inclusion and exclusion criteria by the rules engine (described in a specific example below). Initial eligibility places patients in a monitoring pool for ongoing assessment of eligibility and potential patient benefit. As new diagnostic information is collected by the physician practice, central molecular diagnostic laboratory, and other sources eligibility, potential response to best current evidence-based treatment, and potential benefit from the clinical trial based on genetic-basis of the patients cancer are all presented. On finalization of the patient diagnosis file and final selection of the evidence-based clinical pathway, the physician responsible for the patient's course of therapy is presented with the evidence pathway(s) and prospective clinical trials for which the patient is eligible. A patient portal relates this same information for the patient's review.

The responsible physician and patient make a decision as to whether treatment will be according to the evidence-based treatment pathway or by a clinical trial for which the patient meets the eligibility criteria. Upon the consent of the patient for participation data is transferred to the clinical trial eligibility and confirmation forms. Patient data are made available to a Clinical Data Management System (CDMS), Clinical Trials Management System (CTMS), Clinical Research Data Management System (CRDMS), and Diagnostics and Imaging Workflow System. The system utilizes a Clinical Vocabulary Engine, Document Management System, Authentication and Authorization system for network practice physicians and clinical trial nurses to allow for information to be shared across practices and trials. In this embodiment, the system also comprises Form Building Service, Reporting and Data Extraction System and an Integration Engine.

When the patient is treated using clinical trial protocols, compliance for patients under trial protocol is managed identically to those treated according to Evidence-based Treatment Pathways, wherein compliance is persistently monitored and appropriate alerts issues to the responsible physician, physician in charge of clinical research, and clinical research nurse. In certain embodiments, clinical trial compliance data—as well as other data—are captured in a reporting and data extraction from a central database along with additional data fields as required for the trial design and protocol. In one embodiment, this is done as a pass through to an Electronic Data Capture System. In other embodiments, the data are directly managed for ultimate transmittal to the trial sponsor.

The system utilizes compliance data to determine reimbursement for costs associated with the clinical trial. Patient clinical trial care is authorized through a claims validation procedure in the system and is transmitted to appropriate payer for reimbursement. Other direct trial expense, e.g., therapeutics costs, and validated and then directed to the trial sponsor or their designed clinical research entity for payment. Trial participation termination and trial course of therapy conclusion are all integrated into the trial data management system or collected for formatting into SAS format file for secure transmittal to the trial sponsor or their designee (e.g., external clinical research organization).

In the following specific example, patients having non-small cell lung carcinoma (“NSCLC”) are aligned with appropriate oncology clinical trials based on desired criteria. In this example, inclusion criteria include histologically or cytologically confirmed NSCLC, locally advanced and metastatic disease stage IIIB and IV, evidence of disease progression after one or two cytotoxic treatment regimens, including the use of a platinum agent, and complete recovery from prior chemotherapy side effects to <Grade 2. Further inclusion criteria include patients having at least one uni-dimensional measurable lesion meeting RECIST criteria, ECOG PS 0-2, and patients that are at least 18 years old. In the present example, patients would be required to have adequate organ function, including: adequate bone marrow reserve: ANC>1.5×109/L, platelets>100×109/L, adequate hepatic function (bilirubin<1.5×ULN, AP, ALT, AST<1.5×ULN AP, ALT, and AST<5×ULN) if liver tumor involvement occurs, and proper renal function (creatinin clearance>40 ml/min based on the Cockcroft-Gault formula). Furthermore, exclusion criteria can be based on life expectancy. In some instances, life expectancy must be greater than 12 weeks.

The system can also store exclusion criteria. For instance, patients can be excluded if they are pregnant or lactating women, have medical risks because of non-malignant disease as well as those with active uncontrolled infection, documented brain metastases unless the patient has completed local therapy for central nervous system metastases and has been off corticosteroids for at least two weeks before enrollment, previous treatments with an EGFR-TKI, or in non-squamous histology earlier treatment with pemetrexed and in squamous earlier treatment with docetaxel. Patients can also be excluded if they fail to stop certain medications such as aspirin or other non-steroidal anti-inflammatory agents for a 5-day period. Exclusion criteria can also be based on concomitant treatment with any other experimental drug under investigation. The system stores the inclusion and exclusion criteria from member enrollment sites and places patients into certain trials based on whether patients meet one or more inclusion criteria and whether patients meet exclusion criteria.

The system can utilize daily batch processes to match criteria from patient profiles and clinical trials profiles in the system. The system can utilize technologies incorporating such as Natural Language Processing (NLP) and Dynamic Rules-Workflow Engines to extract clinical data from free-text documents, capture protocol criteria from text documents, and match patients with protocol criteria and rank them in terms of match. Once this has occurred the information is provided to a network member (e.g., healthcare provider). An exemplary format is shown in FIG. 16. The healthcare provider portal contains a list of applicable clinical trials and lists the patients who qualify for these trials. An email alert will be sent whenever updates occur pertaining to new patients who quality for a trial or the instantiation of a new clinical trial.

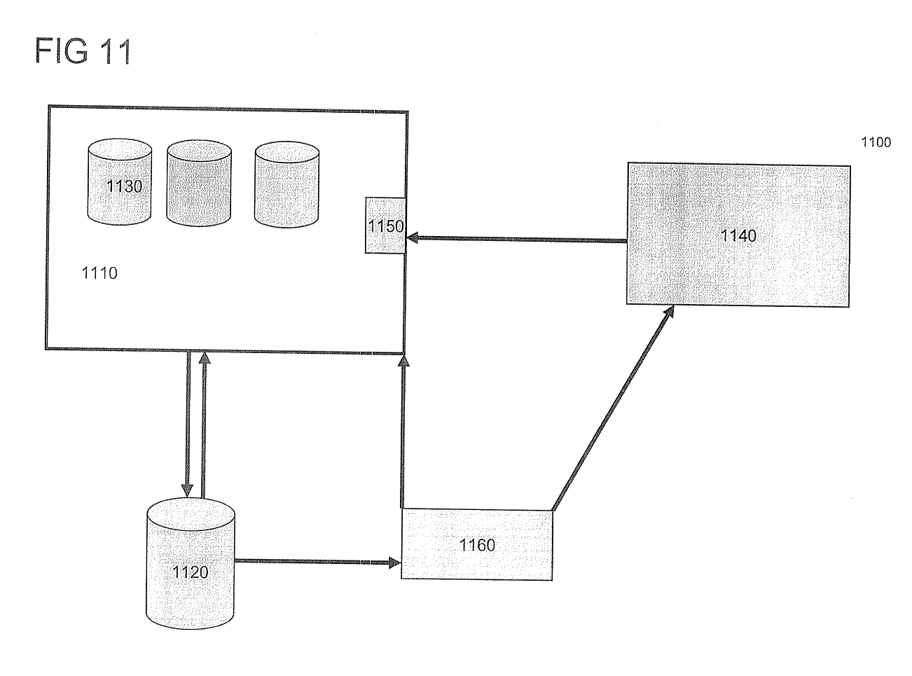

The system also includes the compilation of information from members of a network (“network members”). In some embodiments, “network members” include healthcare practices (referred to in this embodiment as “network member practices”). FIG. 11 shows such an embodiment of the present system 1100. A network member practice 1110 is connected to the KEW database 1120. The network member practice 1110 has electronic medical records (“EMR”) for a patient 1130. The EMR 1130 is provided to the database by the network member practice 1110 as well as any information relating to the diagnosis of the patient. The database 1120 processes the EMR 1130 and information to determine an evidence-based treatment protocol 1160. The pathway is sent to the network member practice 1110. The pathway is utilized by the network member practice 1110 and information regarding treatment is provided to the system 1100. The system 1100 monitors the compliance with the pathway according to the information provided by the network member practice 1110.

In addition, the system 1100 is connected to payers 1140. The network member 1110 submits claims requests 1150 for reimbursement to the system 1100. The system 1100 validates the claim request 1150 based on the network member 1110 compliance to the evidence-based treatment protocol 1160, which is—based on all available information—the most efficacious and cost-effective pathway for the particular patient and disease. The validated claim is forwarded to the payer, who either instructs the system to pay the claim for a designated account or pays the claim directly to the network member practice. Claim reimbursement takes into account compliance to an evidence-based treatment protocol, quality of care associated with the therapy, and payment for the particular therapies used.

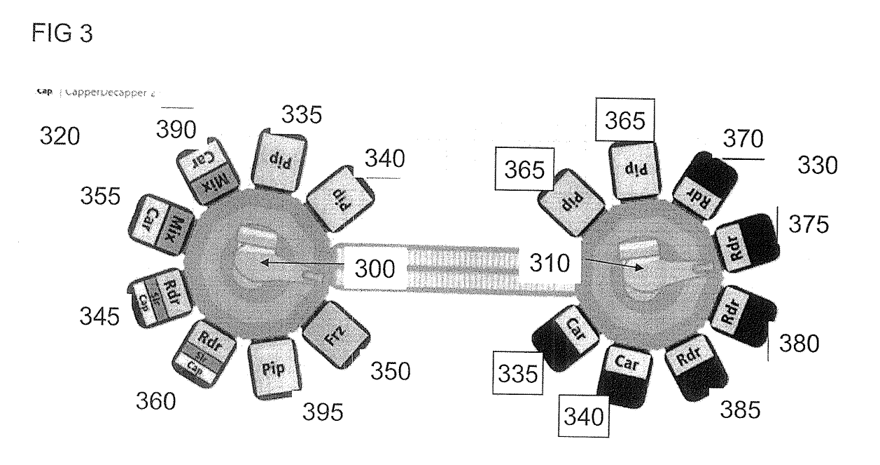

In some aspects, the system includes one or more robot-assisted genomic labs. Robots used in the labs include liquid handling robots such as Biomek laboratory automation workstations (Beckman Coulter). The robot-assisted genomic labs receive a sample from a patient at one of the member healthcare providers. The sample can be blood, interstitial fluid, other secretions, and any tissue that includes cells for the isolation of genomic or proteomic material. The robot-assisted genomic lab is organized to extract the nucleic acids or proteins or other biomarker material from the sample for analysis. FIG. 3 shows the organization of a robot-assisted genomic lab for genomic processing (i.e., the isolation of nucleic acids from a sample and the sequencing of the nucleic acids). Robots 300 and 310 are each positioned in the middle of laboratory work bench space. Each work bench 320 and 330 comprises carts having the experimental materials necessary for processing the genomic or proteomic material. Robot 300 obtains samples and processes the samples to obtain the genomic sample that robot 310 utilizes for sequencing. Robot 300 places a tissue (fresh frozen, formalin fixed paraffin-embedded (FFPE), or otherwise similarly processed) or a blood sample into cart 335 or cart 340. The tissues are processed utilizing a Biomek® FX^{P} Laboratory Automation Workstation (Beckman Coulter). Nucleic acid is extracted from the samples carts 335 or 340 and quality control is performed on the nucleic acids in cart 345. The quality control is performed using real-time quantitative PCR (qPCR), spectrophotometric analysis (optical density, OD260/280), and/or fluorometric method (Hoescht, PicoGreen). An appropriate quantity of nucleic acid is prepared for analysis in carts 335 and 340 while aliquots of the nucleic acids are placed in both long and short-term cold storage in carts 350 and 355, respectively.

The nucleic acid samples are prepared for sequencing. Cart 360 contains the materials for amplifying the region of interest using PCR. In addition, cycle sequencing products are generated with randomly terminated, differentially fluorescent ends. The cycle sequencing set up occurs in cart 365 and is performed in cart 360. The cycle sequencing products are separated by capillary electrophoresis and imaged. In certain embodiments, Sanger sequencing is performed in carts 370-385. The sequencing is performed using standard dideoxynucleotide synthesizing protocols. In certain embodiments, array-based sequencing is performed by amplifying the DNA (cart 360) and fragmenting and end-labeling the region of interest (cart 365). The amplified, labeled region of interest is hybridized to an immobilized target sequence. Other sequencing techniques include SOLiD sequencing or other second, third generation, or post-light sequencing platforms. SOLiD sequencing involves shearing genomic DNA (carts 355 and 390) using a Covaris E210 (Covaris, Inc.)

In certain embodiments, SOLiD libraries and quality control sample prep are generated (cart 395). SOLiD libraries are quantified (cart 345). Libraries are enriched and pooled (cart 395).

The raw sequence data is obtained. The raw sequence data is filtered for quality and aligned to a human genome reference sequence. A module for aligning nucleic acid sequences includes BWA, Picard, and Samtools. After alignment, a module such as Genome Analysis ToolKit (GATK, Broad Institute, open source) or NextGENe (SoftGenetics) identifies changes in the patient's sequence from a human reference sequence (i.e., a consensus sequence for a particular gene or segment of the genome or consensus genome). Any changes (i.e., mutations) from the reference sequence that meet threshold criteria (allelic ratio and others) as well as individual positions for which no or poor quality sequence data have been obtained are documented and stored in the database in a BAM file. The new sequence data is then used to generate hypotheses relating to the effect of the newly identified mutation on the patient's disease. In certain embodiments, the mutation(s) present are queried against the one or more databases that capture the clinical significance of these variants, e.g. benign or pathogenic or responsive or resistant to treatments. If the variant does not exist in the database, the system provides a best guess as to its clinical significance, which could include unknown significance. In addition, the newly identified mutation can be accessed by members of the network for research and analysis.

The system also comprises a module to analyze changes to a patient's genome. The system queries any changes against known human variation and, when known, an assessment of its impact on either prognosis, impact on therapeutics, or other influence on care will be made. The system compares any changes with information from public databases that catalog human genetic variation (dbSNP, COSMIC), published literature, and other sources of information. The changes are also compared with variations stored in the database and with the information obtained from member patients. Changes are reported using standard genetic nomenclature so can be cross-referenced with databases. The database is scanned for information about the disease, treatment response, and outcome of these other patients having the same changes, all of which are used to determine a therapeutic pathway for the patient providing the sample. This is done on the basis of phenotypic and genotypic information that is derived from the patient database as derived from the patients EMR to look for the closest match with clinical relevance and utility. If the variant is commonly observed in the healthy population, it is regarded as a benign finding. If the variant has been shown to have an association with disease or response to drugs this will be reported to members having an interest (i.e., those treating patients with the disease or the member healthcare provider treating the patient supplying the genomic material). For example, a prediction could be made as to the impact a mutation has on the normal function of a protein and therefore its impact on response to therapy. In a specific example, activating mutations in KRAS are known to predict lack of response to EGFR-targeted therapy. If a novel mutation in KRAS was identified, and this variant was predicted to lead to an activation of KRAS similar to other known mutations, the patient could be directed to a non-EGFR-targeted therapy pathway.

There will be instances in which a novel sequence change (i.e., mutation) is identified. If the mutation is previously unknown, a prediction about its potential impact will be made through an assessment process. The assessments are based on principles such as evolutionary conservation of an amino acid at a particular position in homologous proteins in distantly related species or changes of the 3-dimensional structure in a mutant protein. The system includes logics that have been previously reported such as, for example, PolyPhen, SNAP, and SIFT. In addition, the database is scanned for family information. If none exists, samples can be obtained from family members and genomic analyses performed and the information stored in the database. If the mutation occurs in the germline of an individual with a family history of disease, a hypothesis is generated and further testing is suggested. Any additional data generated to support the hypothesis will be stored and the hypothesis evaluated to determine if it aligns with a proven evidence-based protocol.

In additional aspects, the system comprises a module for monitoring the compliance of practitioners with therapeutic pathways. The system identifies the compliance of the practitioner and reports the compliance to the healthcare provider and/or insurer. The system can also calculate the reimbursement of practitioners based on their compliance with the therapeutic pathways calculated by the system. For example, the greater the compliance rates of a practitioner with therapeutic pathways, the higher the reimbursement that the practitioner receives. The system, thus, increases the likelihood that the most efficacious and cost-effective treatments are used by the practitioners in the network.

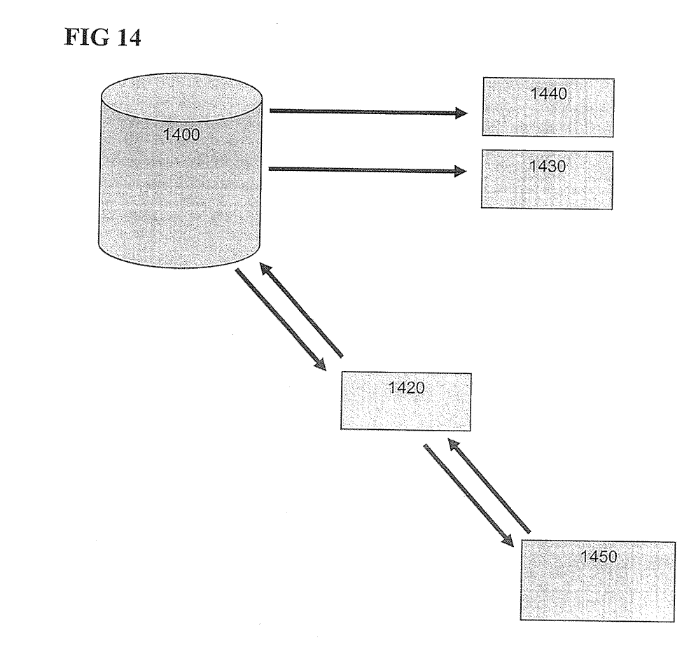

FIG. 14 shows the process of the system 1400 obtaining electronic medical records 1410. Each network member healthcare provider 1450 obtains patient information 1420 such as treatment information, pathology data, and disease information. There are portals that integrate the system and healthcare provider to provide information at the point of care and point of decision making. Furthermore, the system 1400 follows the patient and clinician during the encounter process. The system 1400 further compiles data relating to the patient schedule 1430, drugs and immunizations 1440, and information relating to the patients general health state. Such information is provided through network portals that connect the various remote clinical practices to the system 1400.

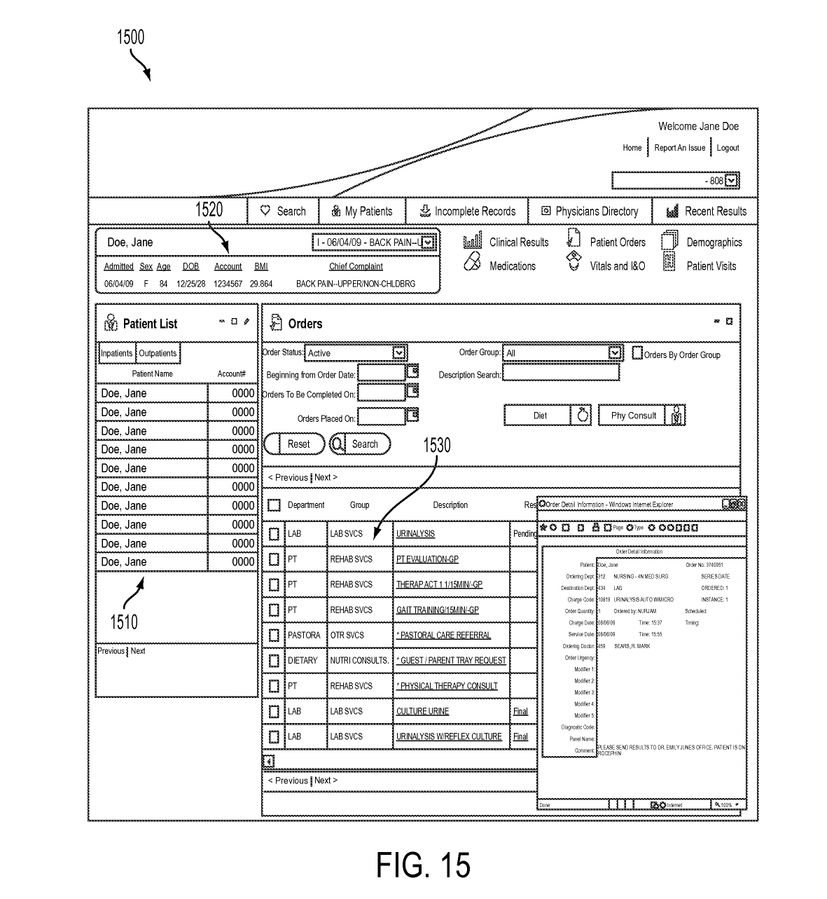

In FIG. 15, the healthcare provider examines a patient and retrieves EMRs and other information on his computer. FIG. 15 shows a screenshot 1500 of the information provided to the provider. The provider receives the patient name 1510 and the account number 1520. The provider also receives the lab results of tests performed on the patient 1530. Furthermore, the clinical complaint of the patient is also stored in the system and sent to the provider. The information retrieved is from a combination of systems. The healthcare provider uses mobile communications or handheld devices to provide information to the system. The healthcare provider can bring this information and other information from the system up on a user interface. Data can be entered in any user interface and such information can be stored in the system and accessed as well. Speech recognition, handhelds and tablets can all be used to enter and retrieve information.

### Calculation of Therapeutic Pathways

Disclosed herein are methods of calculating a therapeutic pathway for a patient suffering from a disease. The methods allow the patient to receive the most efficacious and cost-effective treatments possible based on information. The pathways are generated based on information stored in one or more databases.

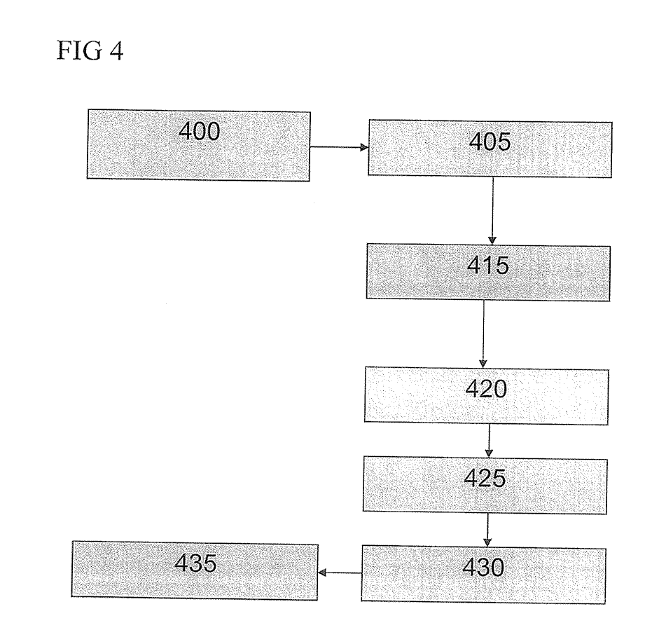

FIG. 4 shows an exemplary method of calculating a therapeutic pathway. Patient data is entered into the system by, for example, a practitioner at a network member 400. The information may be provided manually, or uploaded automatically from data obtained by a network member. Patient data includes both genetic and clinical information relating to the patient. The patient data can include DNA or RNA analyses performed on a patient having a disease. The disease can be an infectious disease (e.g., tuberculosis, HIV, etc.) or it can be a genetic disease such as cancer.

The patient data is obtained through tests and patient consultations. The information is stored in one of the databases in the system 405. In addition to being stored in the system, the information is semantically normalized to the other data. An electronic diagnosis is generated and reviewed by clinicians 410. The clinicians can be specialists in a particular field. The electronic diagnosis is reviewed to ensure the accuracy of the diagnosis. Tests (if any) identified by the electronic diagnosis are performed 415 and the results are reviewed by a specialist. After the results are reviewed, the system generates a therapeutic pathway 420 and the pathway is implemented by the practitioner. Even after the therapeutic pathway is generated and implemented, the system continually monitors the progress of the treatment and adjusts the treatment accordingly 425. The treatment plans (e.g., evidence-based treatment protocols) are represented in a data model in its critical components: diagnosis (ICD-9 classification or other as appropriate to regulatory or reimbursement standards), tumor stage, morphology, line of therapy, and performance status (ECOG). In certain cases, new information is provided to the system 430 and the system recalculates the pathway 435 based on the new information. In certain embodiments, patient information obtained from a general practitioner can be provided by the system upon request of a specialist, such as an oncologist. The specialist uses the information to schedule the patient visit and request any tests that need to be done before patient's initial consultation with the specialist.

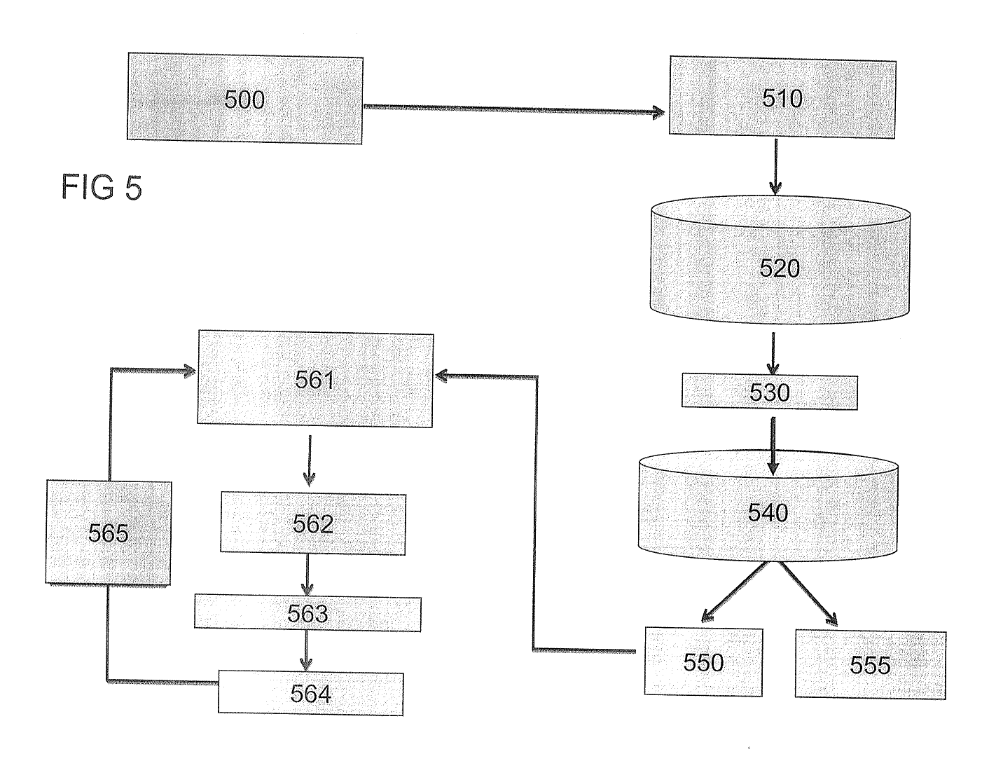

FIG. 5 shows another method of determining the proper therapeutic pathway for a patient in which the system obtains a biological sample. A patient sample is obtained and provided to the system 500. A robot-assisted genomic lab receives the sample and generates a genetic profile from a tissue source 510. The genetic profile is aligned with reference DNA sequences and mutations are identified 520. The genetic profile can be determined using the automated genetic analysis described herein. Once the genetic profile has been identified, it is stored in a database. The system compiles the genetic profile and other information such as the patient's medical history into the database. A determination is made as to whether the mutation is pathologic or benign based on other information compiled in the database or by in silico assessments described above 530. The information provided in the database is analyzed for variations (i.e., mutations) in the genetic profile 540. The system and specialists then determine whether a particular variation is pathological 550 or benign 555. This comparison of the patient's profile to other information in the database allows for the system to identify a pattern in the publicly available information and associate the pattern with the genetic profile of the patient. Such an association is used to determine the outcome of particular courses of treatment and to calculate a therapeutic pathway 560. The system is configured to characterize the disease or risk state of the patient 561. Such a characterization allows for the determination of outcomes. Once the outcomes have been determined, the system will provide information on therapeutic treatments and/or lifestyle recommendations 562. The system will also accumulate information relating to the lifestyle of the patient or the treatment of the patient (i.e., accumulate information relating to the actual outcome) 563. The system also accumulates information relating to particular outcome measures based on patient compliance with the treatment regime and lifestyle changes for the patient 564. The pathway is provided to the practitioner at the network member, in this case, a user of the system, and the therapeutic pathway guides treatment of the patient's disease. The therapeutic pathway can include suggestions on the alteration of lifestyle as well as suggestions on proper therapeutics 565. In certain instances, the outcome reinforces the treatment pathway chosen for the particular disease. The system suggests the therapeutic pathway based on one or more suggested actions predicted to be more likely to yield a positive and cost-effective outcome for the patient.

The progress of the therapeutic pathway is monitored, as well as the outcome, and such information is stored in the system. The monitoring is based on new information that is compiled into the database and determinations of outcomes during treatment of the patient. In the event that a therapeutic pathway (in this instance, an evidence-based treatment protocol) is determined to be impacted by new data, the system recalculates the pathway and alerts the user to the new pathway.

In one specific embodiment, the system retrieves relevant clinical data from electronic medical records (“EMRs”) to suggest a treatment pathway for a patient with stage IV non-small cell lung carcinoma (“NSCLC”) that has been previously untreated. As described below, the system searches information in its database and publicly available information to identify the proper therapeutic pathway. To perform these functions, the system can use the Orion Rhapsody Integration Engine (Orion Health, Santa Monica, Calif.). Other interfaceware solutions can be used, in particular, solutions that support CCD standards.

In this embodiment, the system further reviews the information in the database to derive additional required diagnostics from the EMRs, as well as data retrieved, defined protocols, and empty fields. In the case where additional information is required, the system determines that additional panel tests are required for a determination of the proper therapeutic pathway for the patient having NSCLC. The system suggests such tests and orders the tests. In this embodiment, the system utilizes integrating tools for structured data and unstructured data analysis such as Apixio, MedLEE, and M*Modal.

The system orders tests relating to identifying the genetic background of the NSCLC. The system additionally analyzes the genetic information provided from the genetic tests, particularly analyzing the appropriate region of DNA for NSCLC. The system also searches for changes from a reference sequence (identified from information in the database) that are not known to be benign (also from information in the database). In this embodiment, the system uses a therapeutic protocol based on the NSCLC disease state. This protocol requires sequence information for EGFR, KRAS, BRAF, PIK3CA, and HER2 markers. Furthermore, the copy number information for ALK, HER2, and MET can be used in the protocol. Such sequences are acquired from NCBI. Additionally, such information is acquired from internally generated data of sequence changes. The system can include database support such as Collabrx, GNS healthcare, and Simulconsult.

The system can also compile overall genetic test results and clinical information to begin a query for therapeutic pathway options. For instance, the system determines that the ALK marker is amplified. For instance, the system can use clinical decision support system (“CDSS”) computer software programs. Such programs use Bayesian knowledge-based representations that show a set of variables and their probabilistic relationships between diseases and symptoms. In other embodiments, the programs are based on a rule-based system that captures knowledge that are evaluated by known rules. For example, the clinician can create a rule such as “if the patient has high cholesterol, then the patient is at risk for heart attack.” Accordingly, the system utilizes the rule to make determinations on the importance of tests.

In this embodiment, the system contains a search list of approved therapies for those matching clinical and genetic data identified above. In this embodiment, the therapeutic pathways identified indicate that a first line metastatic NSCLC protocol should be used. The system uses pattern matching algorithms such as Knuth-Morris-Pratt, Boyer-Moore, Text-partitioning, Aho-Corasick, Commentz-Walter, Baeza-Yates, Wu-Manber, and Seminumerical algorithms.

### EQUIVALENTS

There are certain situations in which a patient may be eligible for a therapeutic pathway that comprises inclusion in a clinical trial. In such situations, the system searches a clinical trial registry and determines the eligibility of the selected pathway. The system comprises a database of all trials that are registered. Alternatively, the system can search the database of clinical trials available on the world wide web at clinicaltrials.gov. The system identifies and matches the patient to the proper clinical trial in the event that those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A method of assigning therapeutic pathways to members of a network of oncology treatment providers, the method comprising:
compiling patient data from a network of oncology providers into one or more databases;
compiling publicly available information into the one or more databases;
integrating the patient data and publicly available information into a data set having normalized semantics;
identifying a pattern from a comparison of the patient data to the publicly available information;
calculating a therapeutic pathway based on the pattern, and
providing the therapeutic pathway to a user and monitoring the outcomes.

2. The method of claim 1, wherein the publicly available information is obtained from one or more of clinical trials, university research laboratories, network members, cancer centers, and government research laboratories.

3. The method of claim 1, wherein the publicly available information is obtained from one or more of national cancer registries, FDA databases, genomic databases, and databases administered by the National Institutes of Health.

4. The method of claim 1, wherein the publicly-available information is obtained from payers, patient health records, and employers.

5. The method of claim 1, wherein the publicly-available information comprises information from health record accounts, claim information, self reported information, data related to the clinical information preference, and publicly-available information from other sources.

6. The method of claim 1, wherein the publicly available information comprises genetic information, phenotypic information, genetic profiles, correlations of genetic profiles to disease phenotypes, disease prognoses for genetic profiles, and therapeutic outcomes determined for available therapies.

7. The method of claim 1 further comprising alerting the network to new information relating to the therapeutic pathway.

8. The method of claim 7 further comprising recalculating the therapeutic pathway for the patient based on the new information.

9. The method of claim 1 further comprising tracking compliance of the user to the calculated pathway.

10. The method of claim 9 further comprising calculating the reimbursement of the user based on the compliance with the pathway.

11. The method of claim 1 further comprising obtaining a sample from one or more patients and determining one or more genetic profiles of the one or more patients from one or more tissue sites.

12. The method of claim 11 further comprising compiling the one or more genetic profiles into the one or more databases.

13. The method of claim 12 further comprising identifying one or more patterns from a comparison of the one or more genetic profiles to the publicly available information.

14. The method of claim 11, wherein the patient data comprises one or more genetic profiles and the medical histories of the one or more patients.

15. The method of claim 1, wherein the publicly available information comprises clinical research data.

16. The method of claim 1, wherein the publicly available information comprises data obtained from clinical trials.

17. The method of claim 1 further comprising monitoring a therapeutic outcome of the therapeutic pathway.

18. The method of claim 17 further comprising updating the one or more databases with the therapeutic outcome associated with the therapeutic pathway.

19. The method of claim 18 further comprising recalculating the therapeutic pathway based on the therapeutic outcome and providing the recalculated pathway to the members.

20. The method of claim 1 further comprising compiling financial data from the user.

21. The method of claim 20, wherein the financial data is integrated into the data set.

22. The method of claim 20, wherein the financial data comprises costs associated with the care of the patient.

23. The method of claim 1 further comprising tracking costs associated with the care of the patient.

24. The method of claim 1, wherein the calculating of the therapeutic pathway comprises generating an evidence-based treatment protocol.

25. The method of claim 1 further comprising organizing oncology practices into regional networks.

26. The method of claim 25 further comprising organizing the regional networks into a national oncology network.

27. The method of claim 1, wherein identifying a pattern from a comparison of the patient data to the publicly available information comprises recognizing a pattern in the information and associating the pattern with the patient data.

28. The method of claim 1, wherein the therapeutic pathway guides treatment of one or more patients.

29. The method of claim 1 further comprising analyzing a DNA sequence for at least one region of DNA from a patient or tissue source obtained from a patient.

30. The method of claim 29, wherein a plurality of genes are analyzed.

31. The method of claim 30 further comprising identifying a variation or set of variations in the DNA sequence of the patient as compared to a reference DNA sequence.

32. The method of claim 31 further comprising querying the one or more databases to identify evidence establishing a relationship between the variation or set of variations and one or more of a disease, a therapeutic outcome, or a disease prognosis.

33. The method of claim 32 further comprising generating a hypothesis based on the variation or set of variations and evidence of the relationship.

34. The method of claim 30 further comprising identifying a previously unknown variation or set of variations and compiling these in the one or more databases.

35. The method of claim 34 further comprising producing evidence of a relationship between the unknown variation and one or more of a disease, a therapeutic outcome, or a disease prognosis.

36. The method of claim 35 further comprising providing the evidence to the user.

37. The method of claim 1, wherein the publicly available information comprises the existence of one or more clinical trials testing one or more therapies.

38. The method of claim 37 further comprising identifying one or more clinical trials for which a patient qualifies.

39. The method of claim 37 further comprising creating a cohort of patients for inclusion in a clinical trial based on one or more of genetic and phenotypic information stored in the one or more databases.

40. The method of claim 1, wherein the one or more databases compile structured and unstructured data.

41. The method of claim 1, wherein the databases store digital data that comprise images, sound text, and structured information from electronic medical records.

42. The method of claim 1 further comprising testing for a mutation in one or more genes in a patient or a tissue source derived from a patient, the mutation having a known effect on one or more treatments.

43. The method of claim 1 further comprising testing for a mutation in one or more genes in a patient.

44. The method of claim 43, wherein the mutation has no known effect on a treatment.

45. The method of claim 44 further comprising researching the potential effects of the mutation on one or more treatments.

46. The method of claim 1 further comprising compiling patient data from patient health records, payer related data and self reported data.

47. A genomic analysis and therapy knowledge management system comprising:
one or more robot-assisted genomic labs;
a database in communication with the one or more robot-assisted genomic labs, the database configured to store patient data obtained from the genomic labs, publicly available information, and patient-centric information;
logic configured to integrate the patient data and the information into a data set having normalized semantics;
logic configured to identify a pattern from a comparison of the patient data and patient-centric information to the publicly available information;
logic configured to calculate a therapeutic pathway based on the pattern, and
logic configured to display the calculated pathway to a healthcare provider.

48. The system of claim 47 further comprising logic configured to alert the healthcare provider of new information stored in the database relating to the calculated pathway.

49. The system of claim 47 further comprising logic configured to track costs associated with care of the patient.

50. The system of claim 47, wherein the system comprises one or more of NoSQL databases, columnar databases, and object databases.

51. The system of claim 47 further comprising logic configured to display to the healthcare provider information compiled in the database.

52. The system of claim 48 further comprising logic configured to scan the database for the new information.

53. The system of claim 52 further comprising logic configured to recalculate the calculated pathway based on the new information.

54. The system of claim 47 further comprising logic configured to track compliance of healthcare providers with the calculated pathway.

55. The system of claim 54 further comprising logic configured to calculate reimbursements based on healthcare provider compliance with the calculated pathway.

56. The system of claim 47, wherein the publicly available information is obtained from one or more of clinical trials, university research laboratories, network members, cancer centers, and government research laboratories.

57. The system of claim 47, wherein the publicly available information is obtained from one or more of national cancer registries, FDA databases, genomic databases, and databases administered by the National Institutes of Health.

58. The method of claim 47, wherein the publicly available information comprises genetic information, phenotypic information, genetic profiles associated with one or more diseases, correlations of genetic profiles to phenotypes, disease prognoses, and therapeutic outcomes determined for available therapies.

59. The system of claim 47, wherein the patient-centric information comprises health reimbursement accounts, electronic medical records, patient health records, a personal medical history, and family history of the patient.

60. The system of claim 47, wherein the publicly available information comprises genetic and phenotypic information, genetic profiles associated with one or more diseases, correlations of the genetic profiles to prognoses, correlation of genetic profiles to therapeutic outcomes, drug label warnings, and clinical research data.

61. The system of claim 47 further comprising logic configured to devalue information that is determined to be of lower relevance to the genetic profile or the calculated pathway than other information in the database.

62. The system of claim 47 further comprising logic configured to analyze DNA sequence information generated by the robot-assisted genomic labs.

63. The system of claim 62 further comprising logic configured to generate patient data based on the DNA sequence information.

64. The system of claim 63, wherein the patient data comprises a genetic profile.

65. The system of claim 64 further comprising compiling the genetic profile into the database.

66. The system of claim 47 further comprising compiling financial data from the healthcare provider.

67. The system of claim 66, wherein the financial data is integrated into the data set.

68. The system of claim 66, wherein the financial data comprises costs associated with the care of the patient.

69. The system of claim 47, wherein the logic configured to calculate the therapeutic pathway generates an evidence-based treatment protocol.

70. The system of claim 47 further comprising logic to compare the patient data and the patient-centric information to the publicly available information stored in the database.

71. The system of claim 70 further comprising logic configured to recognize a pattern in the publicly available information and associate the pattern with the genetic profile of the patient.

72. The system of claim 47 further comprising logic configured to identify a variation or set of variations in a DNA sequence of a patient or a tissue obtained from the patient as compared to a reference DNA sequence.

73. The system of claim 72 further comprising logic configured to query the database to identify evidence establishing a relationship between the variation or set of variations and one or more of a disease, a therapeutic outcome, or a disease prognosis.

74. The system of claim 73 further comprising logic configured to generate a hypothesis based on the variation and evidence of the relationship.

75. The system of claim 74 further comprising logic configured to identify a previously unknown variation or set of variations and to compile the variation in the database.

76. The system of claim 75 further comprising logic configured to produce evidence of a relationship between the unknown variation and one or more of a disease, a therapeutic outcome, or a disease prognosis.

77. The system of claim 47, wherein the publicly available information comprises an existence of one or more clinical trials testing one or more therapies.

78. The system of claim 77 further comprising logic configured to identify one or more clinical trials for which a patient qualifies.

79. The system of claim 47 further comprising logic configured to create a cohort of patients for inclusion in a clinical trial based on one or more of genetic and phenotypic information stored in the one or more databases.

80. A method of calculating a therapeutic pathway for a patient suffering from a disease, the method comprising:
generating a genetic profile of the patient or a tissue source obtained from a patient;
compiling the genetic profile and a medical history of the patient into a database;
compiling publicly available information into the database;
comparing the genetic profile and the medical history of the patient to the information compiled in the database;
identifying a pattern in the publicly available information and associating the pattern with the genetic profile of the patient;
calculating the therapeutic pathway based on the pattern identified from the comparison, and
providing the pathway to a user, the therapeutic pathway guiding treatment of the disease.

81. The method of claim 80, wherein the therapeutic pathway comprises one or more suggested actions predicted to be more likely to yield a positive and cost-effective outcome for the patient.

82. The method of claim 80, wherein the publicly available information is obtained from one or more of national cancer registries, FDA databases, genomic databases, and databases administered by the National Institutes of Health.

83. The method of claim 80, wherein genetic and phenotypic information, genetic profiles associated with one or more diseases, correlations of the genetic profiles to prognoses, correlation of genetic profiles to therapeutic outcomes, drug label warnings, and clinical research data.

84. The method of claim 80 further comprising monitoring the therapeutic outcome of the therapeutic pathway.

85. The method of claim 84 further comprising compiling new information relating to the therapeutic pathway into the database.

86. The method of claim 85 further comprising recalculating the therapeutic pathway based on the new information.

87. The method of claim 86 further comprising alerting the user to the recalculated therapeutic pathway.

88. The method of claim 80 further comprising monitoring compliance with the therapeutic pathway.

89. The method of claim 80, wherein the medical history of the patient comprises the family medical history and the treatment history of the patient.

90. The method of claim 80, wherein the user is a healthcare provider.

91. The method of claim 80, wherein the therapeutic pathway comprises an evidence-based treatment protocol.

92. The method of claim 80, wherein the disease is a cancer.

93. The method of claim 80 further comprising determining whether a genetic profile contains a mutation that is associated with a pathological condition or is benign.
